(19) United States
(12) Patent Application Publication
(10) Pub. No.: US 2015/0133810 A1
(43) Pub. Date: May 14, 2015

(54) RESPIRATION ASSIST APPARATUS AND RESPIRATORY FUNCTION TEST SYSTEM

(71) Applicant: DENSO CORPORATION, Kariya-city (JP)

(72) Inventors: Rie OSAKI, Anjo-shi (JP); Taiji KAWACHI, Kariya-shi (JP)

(21) Appl. No.: 14/537,950

(22) Filed: Nov. 11, 2014

(30) Foreign Application Priority Data
Nov. 12, 2013 (JP) 2013-233950

(51) Int. Cl. A61B 5/091; A61B 5/00; A61B 5/08

(52) U.S. Cl. CPC A61B 5/091; A61B 5/0816; A61B 5/742
USPC 600/538

(57) ABSTRACT

In an apparatus for assisting respirations of a subject in accordance with a predetermined reference respiration pattern defined as a sequence of reference respirations each having a reference respiratory volume for the subject, a respiration obtaining unit obtains, at a sampling time, an actual respiratory volume of the subject based on an actual respiration of the subject in accordance with the predetermined reference respiration pattern. A storing unit stores variation of the reference respiratory volumes for the subject. An assisting unit generates, based on the actual respiratory volume and the reference respiratory volumes, visual assist information indicative of a respiratory state of the subject relative to the predetermined reference respiration pattern, and provides the visual assist information to the subject.

### CROSS REFERENCE TO RELATED APPLICATIONS

This application is based on and claims the benefit of priority from Japanese Patent Application 2013-233950 filed on Nov. 12, 2013, the disclosure of which is incorporated in its entirety herein by reference.

### TECHNICAL FIELD

The present disclosure relates to apparatuses for assisting respirations of subjects, and systems for testing the respiratory functions of subjects.

### BACKGROUND

There are known respiratory function test apparatuses for testing the respiratory functions of subjects. For example, one of the known respiratory function test apparatuses is disclosed in Japanese Patent Application Publication No. 2007-229101, referred to as “patent publication”.

The respiratory function test apparatus disclosed in the patent publication is designed to guide a subject to breathe, i.e. take air into and expel air from the subject, at several test times using sounds, and measure the volume of air breathed into and out of the subject for each test time. Based on the measured volumes of air at the respective test times, the respiratory function test apparatus is designed to test the respiratory function of the subject.

### SUMMARY

High-accuracy test of respiratory functions necessitate that actual respiratory patterns, i.e. breathing patterns, of subjects should be close to at least one predetermined ideal respiratory pattern, in other words, at least one predetermined reference respiratory pattern. A respiratory pattern is for example defined based on a sequence of respirations, i.e. breaths, more specifically, defined based on respiration times, respiration lengths, respiration intervals, and/or respiratory volumes, of the subjects.

However, the sound guiding method disclosed in the patent publication may make it difficult for a subject to breathe accurately in agreement with the at least one predetermined ideal respiratory pattern. This may result in difficulty

(1) efficiently guiding respirations of subjects; and

(2) improving the accuracy of the respiratory function tests for subjects.

In view of the circumstances set forth above, one aspect of the present disclosure seeks to provide apparatuses for assisting respirations of a subject and systems for testing a respiratory function of a subject, each of which is designed to address the problem set forth above.

Specifically, an alternative aspect of the present disclosure aims to provide

1. such apparatuses, each of which is capable of efficiently assisting respiration of subjects; and

2. such systems, each of which is capable of improving the accuracy of respiratory function tests for subjects.

According to a first exemplary aspect of the present disclosure, there is provided an apparatus for assisting respirations of a subject in accordance with a predetermined reference respiration pattern defined as a sequence of reference respirations each having a reference respiratory volume for the subject. The apparatus includes a respiration obtaining unit that obtains, at a sampling time, an actual respiratory volume of the subject based on an actual respiration of the subject in accordance with the predetermined reference respiration pattern. The apparatus includes a storing unit that stores variation of the reference respiratory volumes for the subject over time. The apparatus includes an assisting unit that generates, based on the actual respiratory volume and the variation of the reference respiratory volumes, visual assist information indicative of a respiratory state of the subject relative to the predetermined reference respiration pattern, and provides the visual assist information to the subject.

According to a second exemplary aspect of the present disclosure, there is provided a respiratory function test system for testing a respiratory function of a subject. The respiratory function test system includes a respiration obtaining unit that successively obtains actual respiratory volumes of the subject based on actual respirations of the subject in accordance with a predetermined reference respiration pattern. The predetermined reference respiration pattern is defined as a sequence of reference respirations each having a reference respiratory volume for the subject. The respiratory function test system includes a storing unit that stores variation of the reference respiratory volumes for the subject. The respiratory function test system includes an assisting unit that generates, based on the actual respiratory volumes successively obtained by the respiration obtaining unit and the variation of the reference respiratory volumes, visual assist information indicative of a respiratory state of the subject relative to the predetermined reference respiration pattern, and provides the visual assist information to the subject. The respiratory function test system includes a respiratory function testing unit that measures, based on the actual respiratory volumes successively obtained by the respiration obtaining unit, at least one measurement item indicative of the respiratory function of the subject.

According to a third exemplary aspect of the present disclosure, there is provided a computer program product for an apparatus that assists respirations of a subject in accordance with a predetermined reference respiration pattern defined as a sequence of reference respirations each having a reference respiratory volume for the subject. The computer program product includes a non-transitory computer-readable storage medium, and a set of computer program instructions embedded in the computer-readable storage medium. The instructions cause a computer to obtain an actual respiratory volume of the subject based on an actual respiration of the subject in accordance with the predetermined reference respiration pattern, and store variation of the reference respiratory volumes for the subject. The instructions cause a computer to generate, based on the actual respiratory volume and the variation of the reference respiratory volumes, visual assist information indicative of a respiratory state of the subject relative to the predetermined reference respiration pattern. The instructions cause a computer to provide the visual assist information to the subject.

According to each of the first to third exemplary aspects of the present disclosure, the subject easily views the visual assist information provided by the assisting unit, thus easily understanding the respiratory state thereof relative to the predetermined reference respiration pattern. This permits the subject to easily adjust a current actual respiratory volume or a future actual respiratory volume according to the provided visual assist information to, for example, make the current actual respiratory volume or a future actual respiratory volume be close to the predetermined reference respiration pattern. This results in efficient assistance of respirations of the subject.

Various aspects of the present disclosure can include and/or exclude different features, and/or advantages where applicable. In addition, various aspects of the present disclosure can combine one or more feature of other embodiments where applicable. The descriptions of features, and/or advantages of particular embodiments should not be construed as limiting other embodiments or the claims.

### BRIEF DESCRIPTION OF THE DRAWINGS

Other aspects of the present disclosure will become apparent from the following description of embodiments with reference to the accompanying drawings in which:

FIG. 1 is a block diagram schematically illustrating an example of functional blocks of a respiratory function test system according to a first embodiment of the present disclosure;

FIG. 2 is a perspective view schematically illustrating an example of the outward appearance of the respiratory function test system illustrated in FIG. 1;

FIG. 3A is a graph schematically illustrating an example of ideal respiratory patterns suitable for a spirometry test according to the first embodiment;

FIG. 3B is a graph schematically illustrating an example of ideal respiratory patterns suitable for a dynamic hyperinflation test according to the first embodiment;

FIG. 3C is a graph schematically illustrating an example of ideal respiratory patterns suitable for a test of FEV1/FVC according to the first embodiment;

FIG. 4A is a flowchart schematically illustrating an example of the procedure of a respiratory-pattern determining task according to the first embodiment;

FIG. 4B is a flowchart schematically illustrating an example of the procedure of a respiration assist task according to the first embodiment;

FIG. 5A is a graph schematically illustrating an ideal respiratory-pattern waveform according to the first embodiment;

FIG. 5B is a graph schematically illustrating a trajectory of connecting leading ends of already plotted pointers and a guide pointer, which are displayed on the graph of the ideal respiratory-pattern waveform according to the first embodiment;

FIG. 6 is a graph schematically illustrating an execution result of the respiration assist task when the spirometry test illustrated in FIG. 3A is selected as a test item according to the first embodiment;

FIG. 7 is a graph schematically illustrating an execution result of a respiration assist task when a dynamic hyperinflation test illustrated in FIG. 3B is selected as the test item according to the first embodiment;

FIG. 8 is a graph schematically illustrating an execution result of the respiration assist task when the dynamic lung compliance test illustrated in FIG. 9A is selected as the test item according to the first embodiment;

FIG. 9A is a graph schematically illustrating an example of ideal respiratory patterns suitable for a dynamic lung compliance test according to the first embodiment;

FIG. 9B is a graph schematically illustrating an example of the ideal respiratory patterns suitable for a dynamic lung compliance test according to the first embodiment;

FIG. 10A is a graph schematically illustrating an example of the progression of actual respiratory volumes of a test subject according to the first embodiment;

FIG. 10B is a graph schematically illustrating the progression of actual respiratory values of a test subject during the same test item of the respiratory function test according to the first embodiment;

FIG. 11A is a flowchart schematically illustrating an example of the procedure of a respiration assist task according to a second embodiment of the present disclosure;

FIG. 11B is a graph schematically illustrating highest and lowest peaks of an ideal respiratory pattern, a trajectory of connecting leading ends of already plotted pointers, and a guide pointer, which are displayed on the graph of the ideal respiratory-pattern waveform according to the second embodiment;

FIG. 12 is a flowchart schematically illustrating an example of the procedure of a respiration assist task according to a third embodiment of the present disclosure;

FIG. 13 is a bar graph schematically illustrating a bar having a length corresponding to an actual respiratory volume, a bar having a length corresponding to a corresponding ideal respiratory volume, and a guide pointer positioned at a future ideal respiratory volume according to the third embodiment;

FIG. 14 is a block diagram schematically illustrating an example of functional blocks of a respiratory function test system according to a fourth embodiment of the present disclosure;

FIG. 15 is a perspective view schematically illustrating an example of the outward appearance of the respiratory function test system illustrated in FIG. 14 and an example of a pulse wave sensor communicably coupled to the respiratory function test system; and

FIG. 16 is a view schematically illustrating a first sector picture having a center angle and an area corresponding to an actual respiratory volume, a second sector picture having a center angle and an area corresponding to an ideal respiratory volume, and a guide pointer positioned at a future ideal respiratory volume according to a modification of the third embodiment.

### DETAILED DESCRIPTION OF EMBODIMENT

Exemplary embodiments of the present disclosure will be described hereinafter with reference to the accompanying drawings. In the drawings, identical reference characters are utilized to identify identical corresponding components.

### First Embodiment

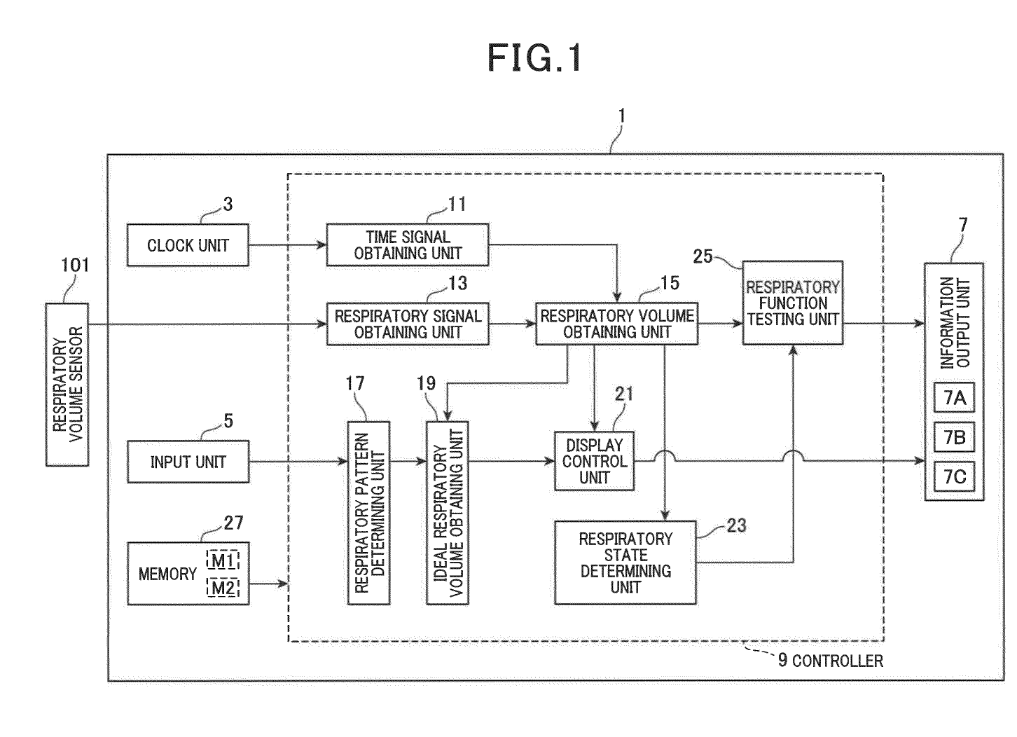

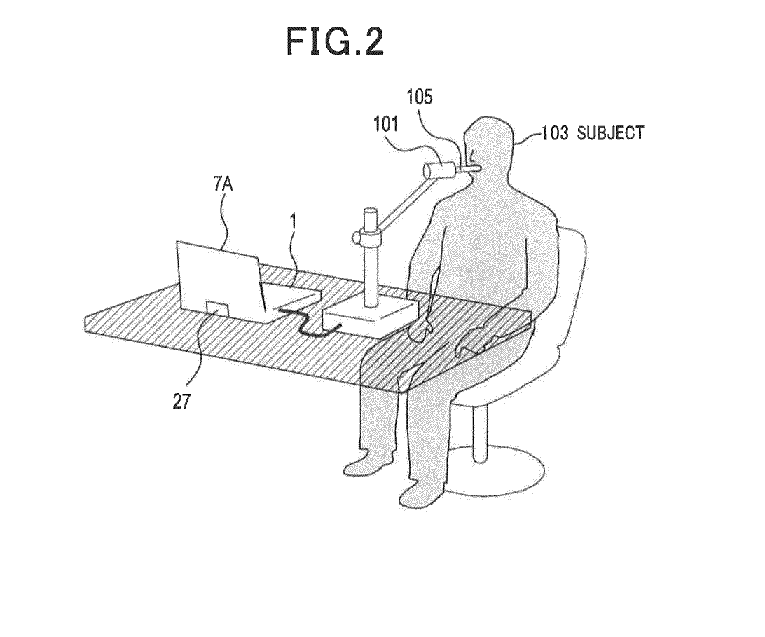

FIG. 1 schematically illustrates an example of the functional blocks of a respiratory function test system 1, which includes a respiration assist apparatus, according to a first embodiment of the present disclosure. FIG. 2 schematically illustrates an example of the outward appearance of the respiratory function test system 1 according to the first embodiment.

The respiratory function test system 1 serves to test the respiratory functions of subjects based on actual respiratory patterns, i.e. breathing patterns, of the subjects. A respiratory pattern of a test subject is for example defined based on a sequence of respirations, i.e. breaths of the test subject, more specifically, defined based on respiration times, respiration lengths, respiration intervals, and/or respiratory volumes, of the test subject.

Specifically, referring to FIG. 1, the respiratory function test system 1 is implemented in, for example, a computer device, such as a personal computer. The respiratory function test system 1 includes a clock unit 3, an input unit 5, an information output unit 7, a controller 9, and a memory serving as a non-volatile storage medium 27.

The clock unit 3 is communicably coupled to the controller 9, and operative to periodically or continuously output a time signal indicative of current time to the controller 9.

The input unit 5 includes one or more input devices, such as, a keyboard, a mouse, a touch panel, and/or various switches; the input devices are communicably coupled to the controller 9. The input unit 5 permits a user to operate the one or more input devices to enter prepared various items of information based on the operation thereinto, and sends the entered various items of information to the controller 9.

The various items of information include, for example, test items, such as a spirometry test, a dynamic hyperinflation test, a test of the FEV1/FVC, i.e. the ratio of forced expiratory volume (%) in 1 second, a dynamic lung compliance test, and the like. The various items of information also include subject information indicative of personal characteristics of a test subject, such as the race, sex, age, height, weight, history of illness, past medical history, and the like about a test subject.

The information output unit 7 includes a display 7A for displaying images, a speaker 7B for outputting sounds, and a vibrator 7C for producing vibratory motions. These devices 7A, 7B, and 7C are communicably coupled to the controller 9. Thus, the information output unit 7 is capable of performing, based on information sent from the controller 9, output of visible information using the display 7A, audible information using the speaker 7B, and vibratory information using the vibrator 7C.

The controller 9 is designed as, for example, a microprocessor, and is communicably coupled to the memory 27 and a respiratory volume sensor 101.

The controller 9 is operative to

1. perform various tasks in accordance with the one or more programs, i.e. instructions, stored in the memory 27 using information input from the clock device 3, the input unit 5, and the respiratory volume sensor 101; and

2. perform information output operations via the information output unit 7 based on the performed tasks.

In the first embodiment, the tasks executable by the controller 9 include a respiratory-pattern determining task and a respiration assist task described later.

The respiratory volume sensor 101 is operative to measure the volume of air breathed into a subject and breathed out of the subject during unit of time for each of the subject's respirations, i.e. for each set of the subject's expiration and inspiration operations. The respiratory volume sensor 101 is operative to output a respiratory signal indicative of the volume, i.e. the respiratory volume, measured for each of the subject's respirations to the controller 9.

Normally, the respiratory signal is designed as a periodic-wave signal that increases during a subject's inspiration operation, and decreases during a subject's expiration operation.

For example, the respiratory volume sensor 101 is disposed in a mouthpiece 105, that is, a tubular member that a subject holds in the mouth. The respiratory volume sensor 101 measures, during unit of time, the volume of air flowing thorough the mouthpiece 105 held by the mouth of a subject (see reference character 103 in FIG. 2) for each of the subject's respirations, i.e. breathings. Then, the respiratory volume sensor 101 outputs the respiratory signal indicative of the measured respiratory volume for each of the subject's respirations to the controller 9.

The controller 9 functionally includes, for example, a time signal obtaining unit 11, a respiratory signal obtaining unit 13, a respiratory volume obtaining unit 15, and a respiratory pattern determining unit 17. The controller 9 also functionally includes, for example, an ideal respiratory-volume obtaining unit 19, a display control unit 21, a respiratory state determining unit 23, and a respiratory function testing unit 25.

The time signal obtaining unit 11 is operative to receive the time signal periodically sent from the clock unit 3, and obtain current time based on the time signal.

The respiratory signal obtaining unit 13 is operative to obtain the respiratory signal sent from the respiratory volume sensor 101.

The respiratory volume obtaining unit 15 is operatively coupled to the time obtaining unit 11 and the respiratory signal obtaining unit 13.

The respiratory volume obtaining unit 15 is operative to integrate the respiratory signal over time determined based on the time signal for each of the subject's respirations, and obtain an actual respiratory volume of the subject in Liters (L) for each of the subject's respirations.

For example, the respiratory volume obtaining unit 15 is operative to obtain, as the actual respiratory volume, an actual volume of air breathed, i.e. inspired, into a subject, and obtain an actual volume of air breathed, i.e. expired, out of the subject for each of the subject's respirations. The actual volume of air inspired into a subject will also be referred to as an actual inspiration volume, and the actual volume of air expired out of a subject will also be referred to as an actual expiration volume.

The clock unit 3, the time obtaining unit 11, the respiratory signal obtaining unit 13, and the respiratory volume obtaining unit 15 serve as a respiration obtaining unit that obtains an actual respiratory volume of a subject based on an actual respiration of the subject in accordance with a predetermined reference respiration pattern.

The respiratory pattern determining unit 17 is operative to

1. determine at least one ideal, i.e. a reference, respiratory pattern for a subject to be tested, and a predetermined respiratory volume for each of predetermined points, i.e. timings, on the determined respiratory pattern based on information input to the controller 9 via the input unit 5; and

2. store the at least one ideal respiratory pattern and the predetermined respiratory volume for each of predetermined points on the determined respiratory pattern.

For example, the at least one ideal respiratory pattern is defined based on respiration times, respiration lengths, respiration intervals, and/or ideal, i.e. reference, respiratory volumes, of a test subject. The detailed functions of the respiratory pattern determining unit 17 will be described later.

In other words, the respiratory pattern determining unit 17 serves as, for example, a storing unit that stores variation of the reference respiratory volumes of a test subject included in an at least one ideal reference respiration pattern.

The ideal respiratory-volume obtaining unit 19 is operatively connected to the respiratory volume obtaining unit 15 and the respiratory pattern determining unit 17. The ideal respiratory-volume obtaining unit 19 is operative to obtain, based on the predetermined respiratory volumes, respective ideal respiratory volumes for a subject to be tested; the respective ideal respiratory volumes should be displayed.

The display control unit 21 is operatively connected to the respiratory volume obtaining unit 15 and the ideal respiratory-volume obtaining unit 19. The display control unit 21 is operative to control how to display, on the display 7A, at least one image that should be displayed on the display 7A of the information output unit 7, and control how to display at least one pointer on an image displayed on the display 7A.

The respiratory state determining unit 23 is operatively connected to the respiratory volume obtaining unit 15, the ideal respiratory-volume obtaining unit 19, and the respiratory function testing unit 25.

The respiratory state determining unit 23 is operative to calculate the deviation of an actual respiratory volume of a test subject obtained by the respiratory volume obtaining unit 11 at each of check timings from an ideal, i.e. a reference, respiratory volume on at least one ideal respiratory pattern determined for the same subject at the same check timing. Then, the respiratory state determiner 23 is operative to output the difference obtained for each of the check timings to the respiratory function testing unit 25.

The respiratory function testing unit 25 is operatively connected to the respiratory volume obtaining unit 15 in addition to the respiratory state determiner 23. The respiratory function testing unit 25 is operative to successively receive the actual respiratory volumes of a test subject for the subject's respective respirations from the respiratory volume obtaining unit 15. Then, the respiratory function testing unit 25 is operative to measure, based on the received actual respiratory volumes of the test subject, at least one predetermined measurement item indicative of the respiratory function of the test subject in accordance with a corresponding at least one algorism predetermined for the at least one measurement item. The predetermined measurement items include, for a test subject, measurement of the lung capacity for the spirometry test; measurement of the dynamic hyperinflation for the dynamic hyperinflation test; measurement of the FEV1/FEV for the test of the FEV1/FVC; measurement of the dynamic lung compliance for the dynamic lung compliance test; and the other similar measurement items.

The respiratory function testing unit 25 is also operative to

1. receive the difference for each of the check timings output from the respiratory state determiner 23;

2. determine whether the difference obtained for each of the check timings is equal to or larger than a predetermined check threshold; and

3. stop measurement of a currently selected measurement item when it is determined that the difference obtained for at least one check timing is equal to or larger than the predetermined check threshold.

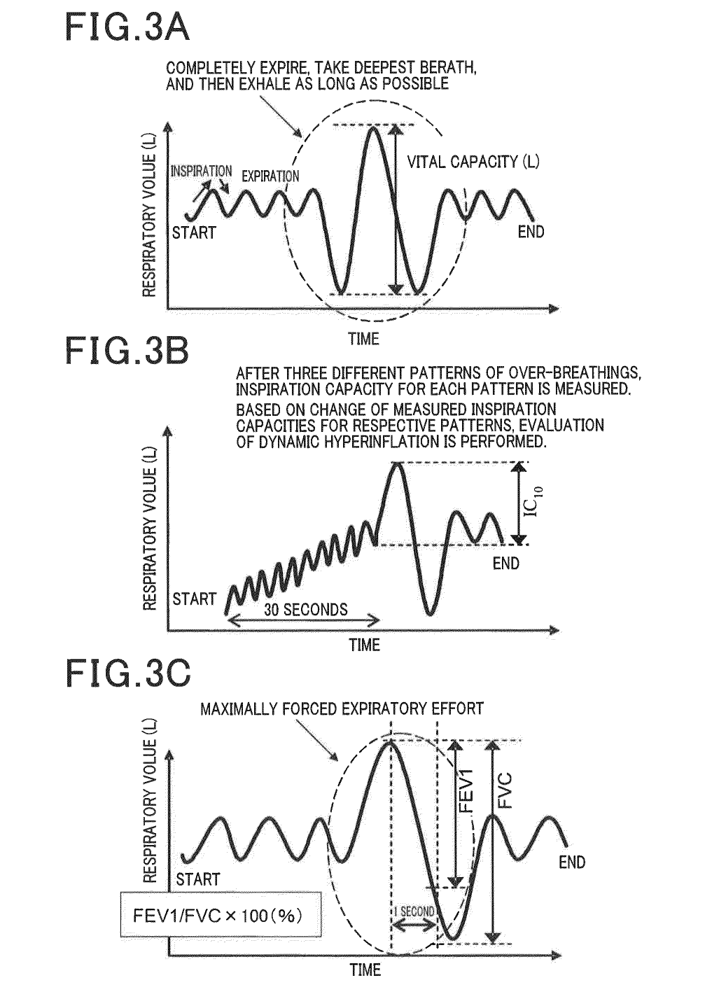

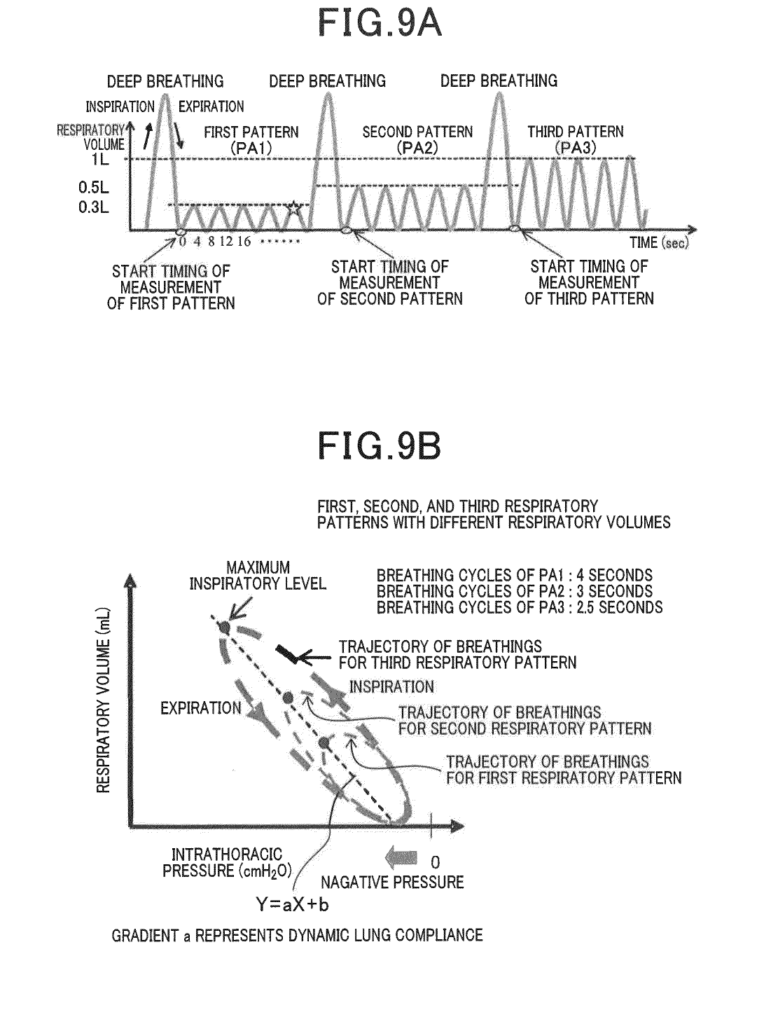

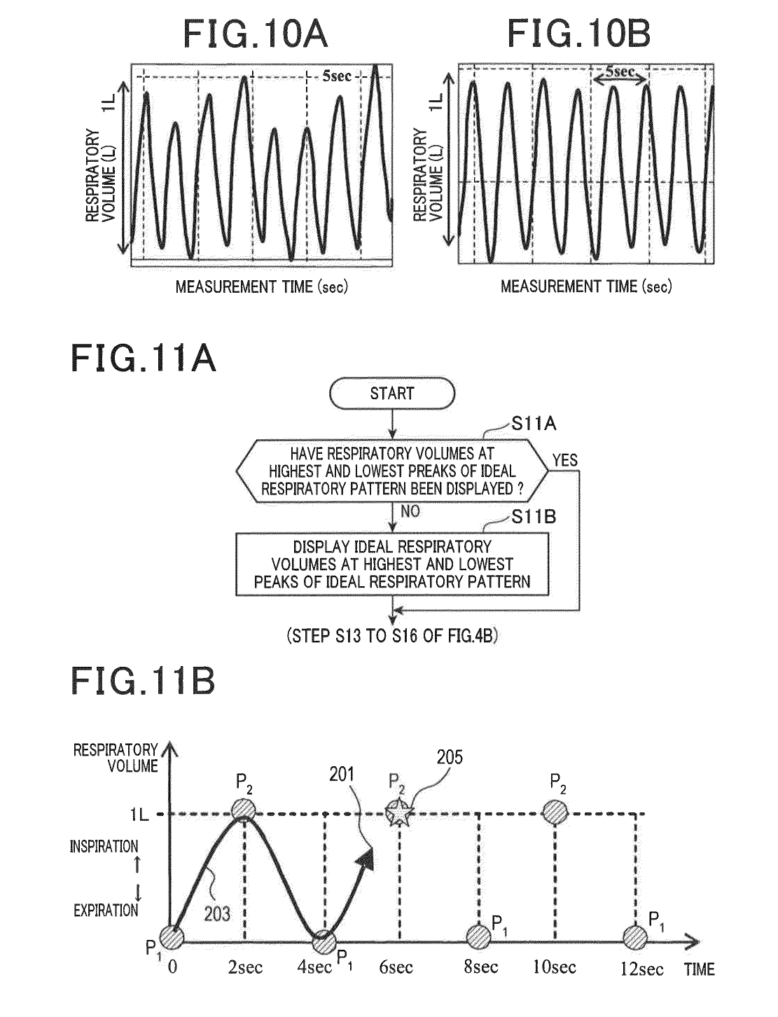

Next, how the controller 9 executes the respiratory-pattern determining task and the respiration assist task will be described hereinafter with reference to FIGS. 3A to 3C, 4A, 4B, and 9A. In each of FIGS. 3A to 3C and 9A, the horizontal axis represents time that has elapsed since start of a corresponding test, and the vertical axis represents a respiratory volume (L) of a test subject. The origin in the vertical axis of each of FIGS. 3A to 3C and FIG. 9A represents the level of a resting respiratory volume of a test subject.

First, specific operations of the controller 9, particularly, the respiratory-pattern determining unit 17, executing the respiratory-pattern determining task will be described. The controller 9 is programmed to execute the respiratory-pattern determining task in response to when test start information is input by an operator thereto from the input unit 5.

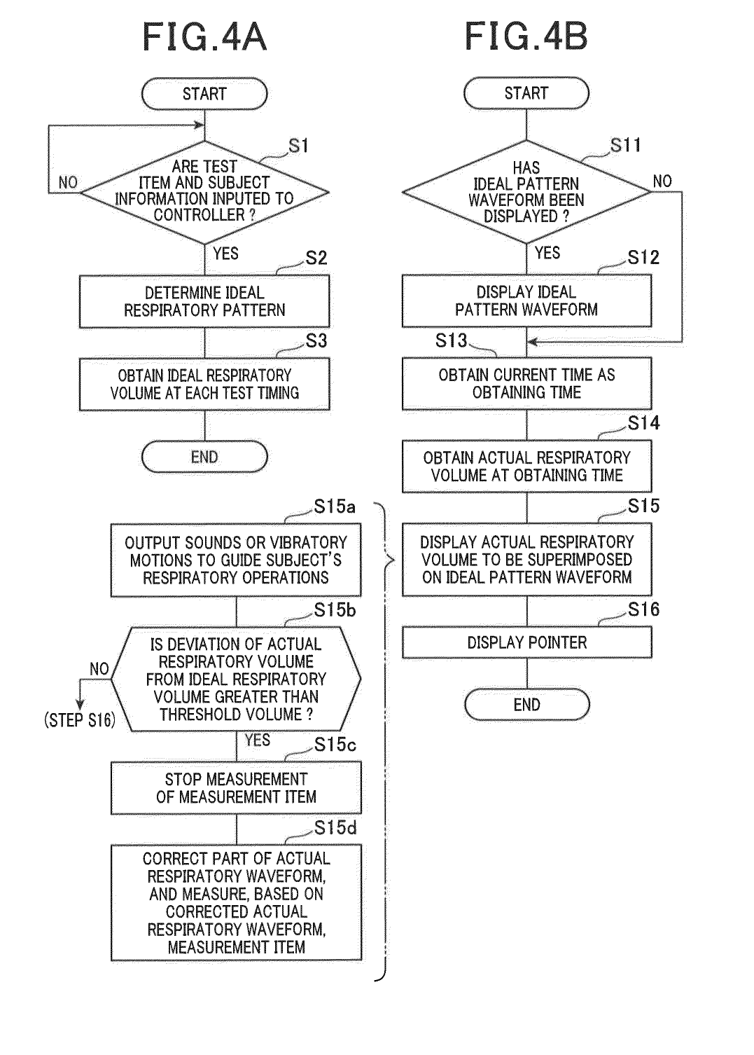

When starting the respiratory-pattern determining task, the controller 9 serves as the respiratory pattern determining unit 17 to repeatedly determine whether a test item and subject information about a test subject are input thereto from the input unit 5 in step S1 of FIG. 4A.

When no test item and subject information are input to the controller 9 (NO in step S1), the controller 9 repeatedly performs the determination in step S1.

Otherwise, when an operator enters a test item and subject information about a test subject into the input unit 5 so that the test item and subject information have been inputted to the controller 9 from the input unit 5 (YES in step S1), the controller 9 determines that the test item and subject information are input thereto from the input unit 5 (YES in step S1). Next, the controller 9 serves as the respiratory pattern determining unit 17 to determine an ideal respiratory pattern suitable for the test item and subject information determined in step S1 in step S2.

In the first embodiment, the memory 27 or the controller 9 stores therein a map M1 representing correlations between each of the test items that can be tested by the respiratory function test system 1 and a predetermined number of ideal respiratory patterns. The predetermined number of ideal respiratory patterns for each test item are prepared for respective items, i.e. the race, sex, age, height, weight, history of illness, past medical history, and the like about a test subject. In the first embodiment, the map M1 is stored in the memory 27 as an example.

That is, in step S2, the controller 9 selects one of the ideal respiratory patterns stored therein or in the memory 27; the selected ideal respiratory pattern match the items of the subject information received in step S1.

For example, when a spirometry test and the subject information are input to the control unit 9, the controller 9 determines an ideal respiratory pattern suitable for the spirometry test and the subject information in step S2. An example of ideal respiratory patterns suitable for the spirometry test is illustrated in FIG. 3A.

FIG. 3A shows that, after a preset number of quiet breathings are performed at substantially constant intervals, i.e. constant breathing cycles, a subject completely expires, takes the deepest breath he/or she can, and then exhales as long as possible. During the spirometry test, the difference between the highest peak and the lowest peak of the waveform of the respiratory signal represents a vital capacity of the subject.

FIG. 3B illustrates an example of ideal respiratory patterns suitable for the dynamic hyperinflation test.

FIG. 3B shows that, after a number of over-breathings are performed at short intervals for preset time, such as 30 seconds, an inspiration capacity is measured. This set is repeated at preset times while changing the number of over-breathings. FIG. 3B shows that, after ten over-breathings are performed during 30 seconds, an inspiration capacity is measured as IC₁₀. Like the inspiration capacity IC₁₀, after twenty over-breathings are performed during 30 seconds, an inspiration capacity is measured as IC₂₀, and after thirty over-breathings are performed during 30 seconds, an inspiration capacity is measured as IC₃₀. Based on the change of the inspiration capacities IC₁₀, IC₂₀, and IC₃₀, evaluation of the dynamic hyperinflation of the subject is performed.

FIG. 3C illustrates an example of ideal respiratory patterns suitable for the test of FEV1/FVC.

FIG. 3C shows FVC (Forced Vital Capacity), which is the vital capacity from a maximally forced expiratory effort, and FEV1, which represents a volume that has been exhaled at the end of the first second of forced expiration. The ratio of the FEV1/FVC can be calculated based on measured FVC and FEV1.

FIG. 9A illustrates an example of ideal respiratory patterns suitable for the dynamic lung compliance test.

FIG. 9A shows that, after deep breathing, a preset number of quiet breathings with a predetermined respiratory volume for each breathing being maintained are performed as a respiratory pattern. This respiratory pattern is sequentially repeated at three times as a first respiratory pattern PA1, a second respiratory pattern PA2, and a third respiratory pattern PA3 while

(i) the respiratory volumes, i.e. the maximum inspiratory levels, of the respective first to third respiratory patterns are increased in the order of the first respiratory pattern, the second respiratory pattern, and the third respiratory pattern; and

(ii) the breathing cycles of the respective first, second, and third respiratory patterns are respectively set to different values of 4 seconds, 3 seconds, and 2.5 seconds.

Note that the dynamic lung compliance test necessitates estimation of a value of the intrathoracic pressure of the test subject for each of the first to third patterns. How to estimate a value of the intrathoracic pressure of a test subject uses one of known estimation methods, an example of which is disclosed in Japanese Patent Application Publication No. 2002-355227. Specifically, the estimation method disclosed in the Patent Publication No. 2002-355227 focuses on the fact that the variation of pulse waves of a test subject highly correlates with the intrathoracic pressure of the test subject. That is, the estimation method includes

1. a step of measuring a pulse wave signal indicative of pulse waves of a test subject;

2. a step of measuring how the pulse wave signal varies; and

3. a step of estimating a value of the intrathoracic pressure of the test subject based on the measured variation of the pulse wave signal.

Then, the dynamic lung compliance test plots the respiratory volumes for the respective first to third patterns on a graph as variation of a vertical axis against variation of the estimated values of the intrathoracic pressure as a horizontal axis (see FIG. 9B). The inventor's research finds the following primary expression [1] between respiratory volume and intrathoracic pressure:

Y=aX+b [1]

where X represents a variation of the estimated values of the intrathoracic pressure, and Y represents a variation of the respiratory volumes for the respective first to third patterns. a is a constant gradient, and b is a constant intercept for the vertical axis. The gradient a represents the lung ability to stretch and expand, or the level of the lung flexibility, i.e. the dynamic lung compliance. The intercept b represents how much volume the test subject expels. In other words, the intercept b represents how easily the test subject expels air.

Next, in step S3, the controller 9 serves as the ideal respiratory volume obtaining unit 19 to

(1) obtain an ideal, i.e. a desired, respiratory volume at each of predetermined test timings determined on the ideal respiratory pattern determined in step S2; and

(2) store the ideal respiratory volume at each of the predetermined test timings determined on the ideal respiratory pattern.

The ideal respiratory volume at each of the predetermined test timings determined on the determined ideal respiratory pattern is required for high-accuracy test using the ideal respiratory pattern.

In the first embodiment, the memory 27 or the controller 9 stores therein a map M2 representing correlations between ideal respiratory volumes and predetermined respective test timings determined on each of the ideal respiratory patterns, all of which are prepared in the map M1.

That is, in step S3, the controller 9 retrieves, from the map M2, ideal respiratory volumes at predetermined respective test timings determined on the ideal respiratory pattern determined in step S2. After completion of the operation in step S3, the controller 9 terminates the respiratory-pattern determining task.

Next, specific operations of the controller 9 executing the respiration assist task will be described. The controller 9 is programmed to cyclically execute the respiration assist task while the test subject actually takes breaths, i.e. respirations, according to the determined test item after the ideal respiratory pattern and the ideal respiratory volumes have been determined in step S2.

When starting the respiration assist task, the controller 9 serves as the display control unit 21 to determine whether an ideal respiratory-pattern waveform 200 visually representing the ideal respiratory pattern and the ideal respiratory volumes at the predetermined respective test timings determined on the ideal respiratory pattern has been displayed as a graph on the display 7A in step S11 of FIG. 4B.

The ideal respiratory-pattern waveform 200 graphically displayed on the display 7A is a visual plotting of the relationship between

1. time, which is shown as the horizontal axis, relative to the start of the respiratory function test; and

2. the ideal respiratory volumes, which are shown as the vertical axis, at the predetermined respective test timings determined on the ideal respiratory pattern.

When it is determined that the ideal respiratory-pattern waveform 200 has been displayed on the display 7A (YES in step S11), the procedure of the respiration assist task goes to step S13. Otherwise, when it is determined that the ideal respiratory-pattern waveform 200 has not been displayed yet on the display 7A (NO in step S11), the controller 9 carries out the operation in step S12. Specifically, in step S12, the controller 9 serves as the display control unit 21 to display the ideal respiratory-pattern waveform 200 on the display 7A based on the ideal respiratory pattern and the ideal respiratory volumes at the predetermined respective test timings determined on the ideal respiratory pattern determined by the respiratory-pattern determining task.

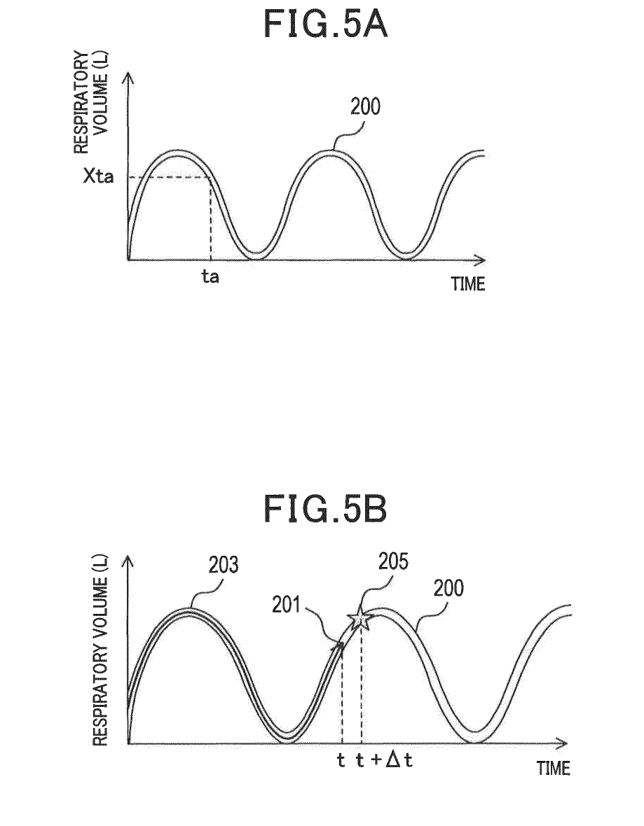

In FIG. 5A, reference character Xta represents an ideal respiratory volume at timing ta determined by the respiratory-pattern determining task. Note that the origin in the graph of FIG. 5A represents the level of a resting respiratory volume of the test subject.

Following the operation in step S11 or step S12, the controller 9 obtains, as obtaining time or sampling time, current time t relative to the start of the respiratory function test based on the time signal obtained by the time signal obtaining unit 11 in step S13.

Next, in step S14, the controller 9 serves as the respiratory signal obtaining unit 13 and respiratory volume obtaining unit 15 to obtain an actual respiratory volume of the test subject at the obtaining time, i.e. sampling time, t obtained in step S13.

Following the operation in step S14, the controller 9 serves as the display control unit 21 to display the actual respiratory volume of the test subject at the obtaining time t on the display 7A such that the actual respiratory volume of the test subject at the obtaining time t is superimposed on the ideal respiratory-pattern waveform 200 displayed on the display 7A in step S15.

In other words, the display control unit 15 serves as an assisting unit that generates, based on the actual respiratory volume of the test subject at the obtaining time t and the ideal respiratory-pattern waveform 200 as an example of the variation, visual assist information. The visual information represents a respiratory state of the subject relative to the ideal respiratory-pattern waveform 200, and provides the visual assist information to the subject via the display 7A.

As an example of the visual assist information, as illustrated in FIG. 5B, the controller 9 plots, on the graph of the ideal respiratory-pattern waveform 200 displayed on the display 7A, a pointer, such as an arrow, 201; the pointer 201 is positioned at the actual respiratory volume of the test subject in the vertical axis and positioned at the obtaining time t determined in step S13 in the horizontal axis.

Because the controller 9 cyclically performs the respiration assist task when the test subject actually starts a respiratory function test, the pointers 201 have been plotted on the graph of the ideal respiratory-pattern waveform 200 displayed on the display 7A. Thus, in step S15, the controller 9 displays a trajectory 203 connecting the leading ends of the already plotted pointers 201 on the graph of the ideal respiratory-pattern waveform 200 displayed on the display 7A while the leading end of the trajectory 203 forms as an arrow 201. The arrow 201 indicates the actual respiratory volume of the test subject at the obtaining time t on the graph of the ideal respiratory-pattern waveform 200 (see FIG. 5B). The arrow 201 also indicates a direction of a moving direction of the trajectory 203.

Note that, in this case, the controller 9 erases an already displayed arrow 201 at the previous cycle of the respiration assist task in step S15.

Following the operation in step S15, the controller 9 serves as the ideal respiratory-volume obtaining unit 19 to obtain a future ideal respiratory volume at future time defined after a lapse of Δt from the obtaining time t on the ideal respiratory pattern in step S16. The future time will be referred to as t+Δt.

In step S16, the controller 9 serves as the display control unit 21 to display a guide marker or pointer 205 positioned at the future ideal respiratory volume in the vertical axis and positioned at the future time t+Δt in the horizontal axis; the guide marker or pointer 205 points out the future ideal respiratory volume. For example, in FIG. 5B, a star-shaped guide marker 205 is displayed on the ideal respiratory-pattern waveform 200. After completion of the operation in step S16, the controller 9 terminates the respiration assist task. As described above, the controller 9 cyclically executes the respiration assist task.

The cyclic execution of the respiration assist task results in the pointer 201 indicating the actual respiratory volume moving in the right direction on the graph of the ideal respiratory-pattern waveform 200 over time. Thus, if the actual respiratory volumes measured based on respirations of the test subject follow the ideal respiratory-pattern waveform 200, the pointer 201 will move on the ideal respiratory-pattern waveform 200. However, if the actual respiratory volumes measured based on respirations of the test subject do not follow the ideal respiratory-pattern waveform 200, the pointer 201 will deviate from the ideal respiratory-pattern waveform 200.

The trajectory 203 follows movement of the pointer 201 to the right on the graph of the ideal respiratory-pattern waveform 200, thus moving in the right direction.

The guide marker or pointer 205 also follows movement of the pointer 201 in the right direction on the graph of the ideal respiratory-pattern waveform 200, thus moving in the right direction while being located on the right side of the pointer 201 by the length of Δt.

Note that the controller 9 serves as the respiratory function testing unit 25 to measure, based on the received actual respiratory volumes of the test subject, a predetermined measurement item corresponding to the test item input from the input unit 5 in step S1 while performing the respiration assist task.

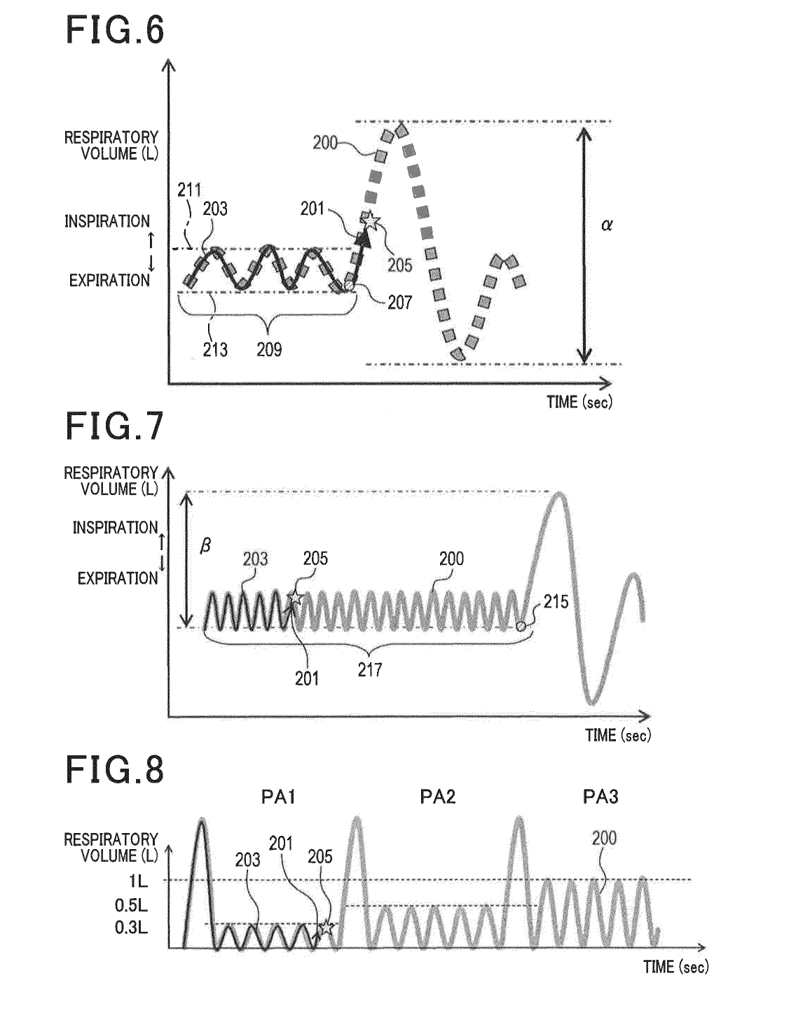

FIG. 6 schematically illustrates an execution result of the respiration assist task when the spirometry test illustrated in FIG. 3A is selected as the test item in step S1.

Referring to FIG. 6, reference character 207 represents start timing of the deepest breath of a test subject, and reference character 209 represents the range of the quiet breathings. Reference numeral 211 represents a top line substantially passing highest peaks of the ideal respiratory-pattern waveform 200 for the spirometry test within the range of the quiet breathings, and reference character 213 represents a bottom line substantially passing lowest peaks of the ideal respiratory-pattern waveform 200 for the spirometry test within the range of the quiet breathings. Reference character α represents a vital capacity of the test subject.

As illustrated in FIG. 6, the arrow pointer 201 is displayed to be superimposed on the ideal respiratory-pattern waveform 200 to indicate, at its leading end, the actual respiratory volume of the test subject at obtaining time t. The trajectory 203 connecting the leading ends of the already plotted arrow pointers 201 is displayed on the graph of the ideal respiratory-pattern waveform 200. In addition, the star-shaped guide marker 205 is displayed on the graph of the ideal respiratory-pattern waveform 200 while indicating the future ideal respiratory volume at the future time t+Δt. Note that the ideal respiratory-pattern waveform 200 illustrated in FIG. 6 schematically illustrates a desired respiratory waveform as a guideline for the test subject, and therefore does not necessarily shows an actual ideal respiratory pattern accurately.

FIG. 7 schematically illustrates an execution result of the respiration assist task when the dynamic hyperinflation test illustrated in FIG. 3B is selected as the test item in step S1.

Referring to FIG. 7, reference character 215 represents start timing of measurement of the inspiration capacity (IC) of a test subject, and reference character 217 represents the duration of the over-breathings. Reference character β represents the inspiration capacity of the test subject.

As illustrated in FIG. 7, the arrow pointer 201 is displayed to be superimposed on the ideal respiratory-pattern waveform 200 to indicate, at its leading end, the actual respiratory volume of the test subject at obtaining time t. The trajectory 203 connecting the leading ends of the already plotted arrow pointers 201 is displayed on the graph of the ideal respiratory-pattern waveform 200. In addition, the star-shaped guide marker 205 is displayed on the graph of the ideal respiratory-pattern waveform 200 while indicating the future ideal respiratory volume at the future time t+Δt. Note that the ideal respiratory-pattern waveform 200 illustrated in FIG. 7 schematically illustrates a desired respiratory waveform as a guideline for the test subject, and therefore does not necessarily shows an actual ideal respiratory pattern accurately.

FIG. 8 is a graph schematically illustrating an execution result of the respiration assist task when the dynamic lung compliance test illustrated in FIG. 9A is selected as the test item according to the first embodiment.

Referring to FIG. 8, reference character PA1 represents the first respiratory pattern with the respiratory volume of 0.3 L, reference character PA2 represents the second respiratory pattern with the respiratory volume of 0.5 L, and reference character PA3 represents the third respiratory pattern with the respiratory volume of 1.0 L.

As illustrated in FIG. 8, the arrow pointer 201 is displayed to be superimposed on the ideal respiratory-pattern waveform 200 to indicate, at its leading end, the actual respiratory volume of the test subject at obtaining time t. The trajectory 203 connecting the leading ends of the already plotted arrow pointers 201 is displayed on the graph of the ideal respiratory-pattern waveform 200. In addition, the star-shaped guide marker 205 is displayed on the graph of the ideal respiratory-pattern waveform 200 while indicating the future ideal respiratory volume at the future time t+Δt. Note that the ideal respiratory-pattern waveform 200 illustrated in FIG. 8 schematically illustrates a desired respiratory waveform as a guideline for the test subject, and therefore does not necessarily shows an actual ideal respiratory pattern accurately.

As described above, during a respiratory function test of a test subject based on a selected test item, the respiratory function test system 1, in other words, the respiration assist apparatus included in the test system 1 is capable of

(i) determining an ideal respiratory pattern based on the selected test item and entered subject information of the test subject and ideal respiratory volumes at predetermined respective test timings determined on the ideal respiratory pattern;

(ii) displaying, on the display 7A, an ideal respiratory-pattern waveform 200 visually representing the ideal respiratory pattern and the ideal respiratory volumes at the predetermined respective test timings determined on the ideal respiratory pattern;

(iii) obtaining an actual respiratory volume of the test subject at each obtaining time; and

(iv) displaying, using a pointer 201, the actual respiratory volume obtained for each of the obtaining times such that the actual respiratory volume 201 is superimposed on the ideal respiratory-pattern waveform 200 displayed on the display 7A.

This capability enables the test subject to visibly compare the actual respiratory volume obtained for each obtaining time with the ideal respiratory-pattern waveform 200, making it possible for the test subject to easily understand the relationship between the actual respiratory volume obtained for each obtaining time, i.e. each sampling timing, and the ideal respiratory-pattern waveform 200.

This capability also permits the test subject to easily understand how the ideal respiratory pattern will change from each of the obtaining times.

This enables the test subject to adjust next actual respiratory time and a next actual respiratory volume after each obtaining time such that the next actual respiratory volume at the next actual respiratory time after each obtaining time matches with the ideal respiratory volume at the same time. This results in an improvement of the accuracy of the respiratory function tests to be carried out by the respiratory function test system 1.

Particularly, the respiratory function test system 1 is capable of displaying a guide marker or pointer 205 indicating a future ideal respiratory volume at future time defined after a lapse of preset time Δt from each obtaining time. This makes the guide marker or pointer 205 follow movement of the pointer 201, i.e. the actual respiratory volume 201, thus making for the test subject to understand the next ideal respiratory volume after each sampling time, which should be required for meeting the ideal respiratory pattern.

Accordingly, the respiratory function test system 1 is capable of efficiently assisting respirations, i.e. breathings, of test subjects, thus improving the accuracy of their respiratory function tests.

FIG. 10A illustrates, as a graph, the progression of actual respiratory values of a test subject during a selected test item of a respiratory function test; the actual respiratory volumes were obtained when the graph illustrated in FIG. 5B is not displayed on the display 7A. Note that the vertical axis of the graph represents the actual respiratory volumes (L), and the horizontal axis represents measurement time (sec) of the actual respiratory volumes.

As illustrated in FIG. 10A, the actual respiratory volumes, the breathing cycles, and actual breathing waveforms vary for respective breathings.

In contrast, FIG. 10B illustrates, as a graph, the progression of actual respiratory values of the same test subject during the same test item of the respiratory function test; the actual respiratory volumes were obtained when the graph illustrated in FIG. 5B is displayed on the display 7A. Note that the vertical axis and the horizontal axis of the graph illustrated in FIG. 10B are the same as those of the graph illustrated in FIG. 10A.

As clearly illustrated in FIG. 10B, the actual respiratory volumes, the breathing cycles, and the actual breathing waveforms are stable, so that there are few variations in each of the actual respiratory volumes, the breathing cycles, and the actual breathing waveforms.

### Second Embodiment

A respiratory function test system, which includes a respiration assist apparatus, according to a second embodiment of the present disclosure will be described hereinafter.

The structure of the respiratory function test system according to the second embodiment is identical to that of the respiratory function test system 1, so that the description of which is omitted.

The functions of the respiratory function test system according to the second embodiment are slightly different from those of the respiratory function test system 1 by the following points. So, the different points will be mainly described hereinafter.

The controller 9 according to the second embodiment is programmed to execute the respiratory-pattern determining task in the same manner as the controller 9 according to the first embodiment.

Next, specific operations of the controller 9 executing the respiration assist task according to the second embodiment will be described. The controller 9 is programmed to cyclically execute the respiration assist task while the test subject actually takes breaths, i.e. respirations, according to the determined test item after the ideal respiratory pattern and the ideal respiratory volumes have been determined in step S2.

Referring to FIG. 11A, the controller 9 determines whether ideal respiratory volumes at respective highest peaks P1 and lowest peaks P2 of the ideal respiratory pattern have been displayed as a graph on the display 7A in step S11A.

As illustrated in FIG. 11B, the highest peaks P1 represent the positions of end-expirations on the ideal respiratory pattern, and the lowest peaks P2 represent the positions of end-inspirations on the ideal respiratory pattern.

When it is determined that the ideal respiratory volumes at the respective highest peaks P1 and lowest peaks P2 of the ideal respiratory pattern have been displayed on the display 7A (YES in step S11A), the procedure of the respiration assist task goes to step S13.

Otherwise, when it is determined that the ideal respiratory volumes at the respective highest peaks P1 and lowest peaks P2 of the ideal respiratory pattern have not been displayed yet on the display 7A (NO in step S11A), the controller 9 carries out the operation in step S12A of FIG. 11A.

Specifically, in step S12A, the controller 9 serves as the display control unit 21 to select, from the ideal respiratory volumes obtained in step S3, the ideal respiratory volumes at the respective highest peaks P1 and lowest peaks P2 of the ideal respiratory pattern. Then, in step S12A, the controller 9 displays the ideal respiratory volumes at the respective highest peaks P1 and lowest peaks P2 of the ideal respiratory pattern on the display 7A (see FIG. 11B).

The other operations of the respiration assist task according to the second embodiment are substantially identical to those of the respiration assist task according to the first embodiment.

Because the ideal respiratory volumes at the respective highest peaks P1 and lowest peaks P2 of the ideal respiratory pattern sufficiently include a schematic waveform of the ideal respiratory pattern, the respiratory function test system according to the second embodiment achieves substantially the same advantages as those achieved by the respiratory function test system 1.

### Third Embodiment

A respiratory function test system, which includes a respiration assist apparatus, according to a third embodiment of the present disclosure will be described hereinafter.

The structure of the respiratory function test system according to the third embodiment is identical to that of the respiratory function test system 1, so that the description of which is omitted.

The functions of the respiratory function test system according to the third embodiment are different from those of the respiratory function test system 1 by the following points. So, the different points will be mainly described hereinafter.

Specific operations of the controller 9 executing the respiration assist task according to the third embodiment will be described. The controller 9 is programmed to cyclically execute the respiration assist task while the test subject actually takes breaths, i.e. respirations, according to the determined test item after the ideal respiratory pattern and the ideal respiratory volumes have been determined in step S2.

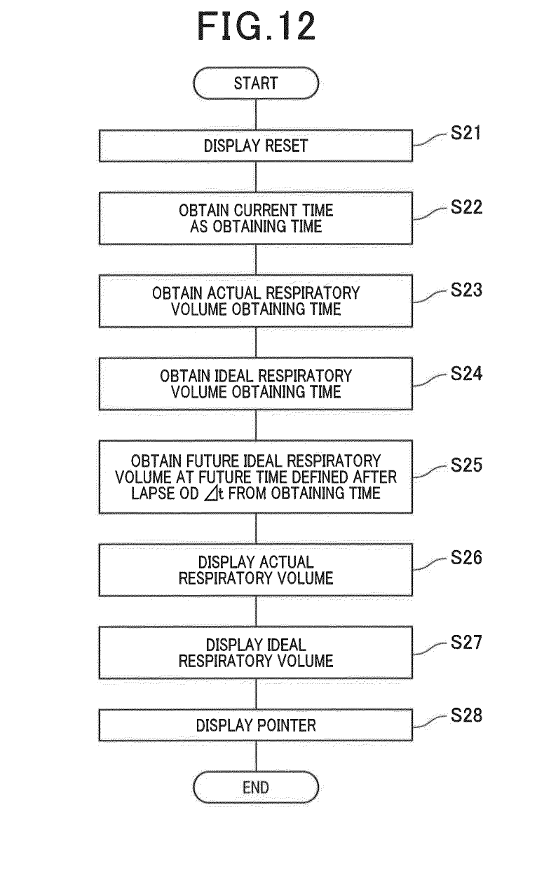

When starting the respiration assist task, the controller 9 serves as the display control unit 21 to reset information displayed on the display 7A in step S21 of FIG. 12.

Next, the controller 9 obtains, as obtaining time, current time t relative to the start of the respiratory function test based on the time signal obtained by the time signal obtaining unit 11 in step S22.

Following the operation in step S22, the controller 9 serves as the respiratory signal obtaining unit 13 and respiratory volume obtaining unit 15 to obtain an actual respiratory volume of the test subject at the obtaining time t obtained in step S23.

Next, in step S24, the controller 9 serves as the ideal respiratory-volume obtaining unit 19 to obtain an ideal respiratory volume at the obtaining time t on the ideal respiratory pattern in step S3.

Following the operation in step S24, the controller 9 serves as the ideal respiratory-volume obtaining unit 19 to obtain a future ideal respiratory volume at future time defined after a lapse of Δt from the obtaining time t on the ideal respiratory pattern in step S25. The future time will be referred to as t+Δt.

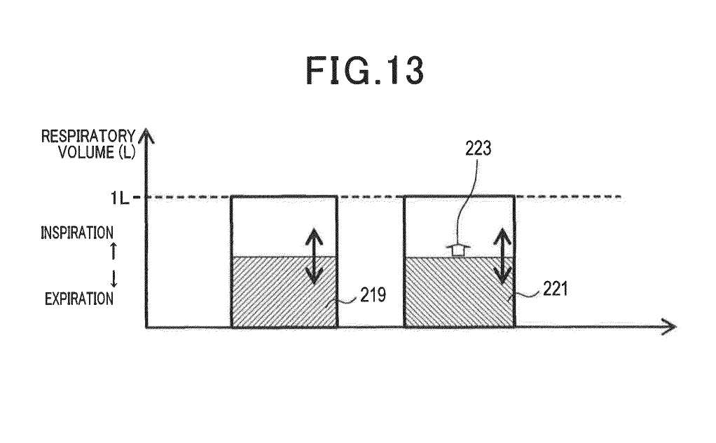

Next, the controller 9 serves as the display control unit 21 to display, on the display 7A, a first bar 219 serving as a first graphic object; the first bar 219 has a length corresponding to the actual respiratory volume obtained in step S23 in the form of a bar graph whose vertical axis represents variation of the actual respiratory volumes in step S26 (see FIG. 13).

Following the operation in step S26, the controller 9 serves as the display control unit 21 to display, on the display 7A, a second bar 221 serving as a second graphic object; the second bar 221 has a length corresponding to the ideal respiratory volume at the obtaining time t obtained in step S24 in step S27. Specifically, the controller 9 displays the second bar 221 at an immediate right side of the bar 219 on the bar graph illustrated in FIG. 13 in step S27.

Next, the controller 9 serves as the display control unit 21 to display, on the bar graph illustrated in FIG. 13, an arrow guide pointer 223 positioned at the future ideal respiratory volume in the vertical axis of the bar graph in step S28. The arrow guide pointer 223 is located above or below the top of the second bar 221 and points out the future ideal respiratory volume in step S28.

For example, if the future ideal respiratory volume is greater than the ideal respiratory volume at the obtaining time t, the arrow guide pointer 223 is located above the top of the second bar 221 and directed upward. If the future ideal respiratory volume is smaller than the ideal respiratory volume at the obtaining time t, the arrow guide pointer 223 is located below the top of the second bar 221 and directed downward. After completion of the operation in step S28, the controller 9 terminates the respiration assist task according to the third embodiment. As described above, the controller 9 cyclically executes the respiration assist task.

The cyclic execution of the respiration assist task results in the length of the first bar 219 changing over time depending on variations of the actual respiratory volumes obtained at the respective obtaining times t. The cyclic execution of the respiration assist task also results in the length of the second bar 221 changing over time depending on variations of the ideal respiratory volumes at the same obtaining times t. The cyclic execution of the respiration assist task further results in the position of the arrow guide pointer 223 rising or falling over time depending on variations of the future ideal respiratory volumes at the future times t+Δt. Additionally, the cyclic execution of the respiration assist task results in the direction of the arrow guide pointer 223 showing an increase or decrease of the trajectory of the ideal respiratory volumes after each obtaining time t.

As described above, during a respiratory function test of a test subject based on a selected test item, the respiratory function test system according to the third embodiment is capable of

1. obtaining an actual respiratory volume of the test subject at each obtaining time;

2. displaying, on the display 7A, the bar graph illustrated in FIG. 13 in which the length of the first bar 219 represents the actual respiratory volume of the test subject at each obtaining time t;

3. determining an ideal respiratory pattern based on the selected test item and entered subject information of the test subject and ideal respiratory volumes at predetermined respective test timings determined on the ideal respiratory pattern; and

4. displaying, on the display 7A, the bar graph illustrated in FIG. 13 in which the length of the second bar 221 represents the ideal respiratory volume at each obtaining time t.

The respiratory function test system according to the third embodiment is further capable of

(i) displaying, on the bar graph, the arrow guide pointer 223 representing the ideal respiratory volume at the future time t+Δt defined after a lapse of the preset time Δt from each obtaining time t; and

(ii) showing, by the direction of the arrow guide pointer 223, an increase or decrease from the current ideal respiration volume at each obtaining time t.

These capabilities permit the test subject to visibly compare the length of the first bar 219 with that of the second bar 221 for each obtaining time t, making it possible for the test subject to easily understand the relationship between the actual respiratory volume and the ideal respiratory volume for each obtaining time t.

These capabilities also permit the test subject to visibly recognize the direction of the arrow guide pointer 223, making it possible for the test subject to easily understand how the ideal respiratory pattern will change from each obtaining time t.

This enables the test subject to adjust next actual respiratory time and a next actual respiratory volume after each obtaining time t such that the next actual respiratory volume at the next actual respiratory time after each obtaining time t matches with the ideal respiratory volume at the same time. This results in an improvement of the accuracy of the respiratory function tests to be carried out by the respiratory function test system according to the third embodiment

### Fourth Embodiment

A respiratory function test system 1A according to a fourth embodiment of the present disclosure will be described hereinafter.

The structure and functions of the respiratory function test system 1A according to the fourth embodiment are different from those of the respiratory function test system 1 by the following points. So, the different points will be mainly described hereinafter.

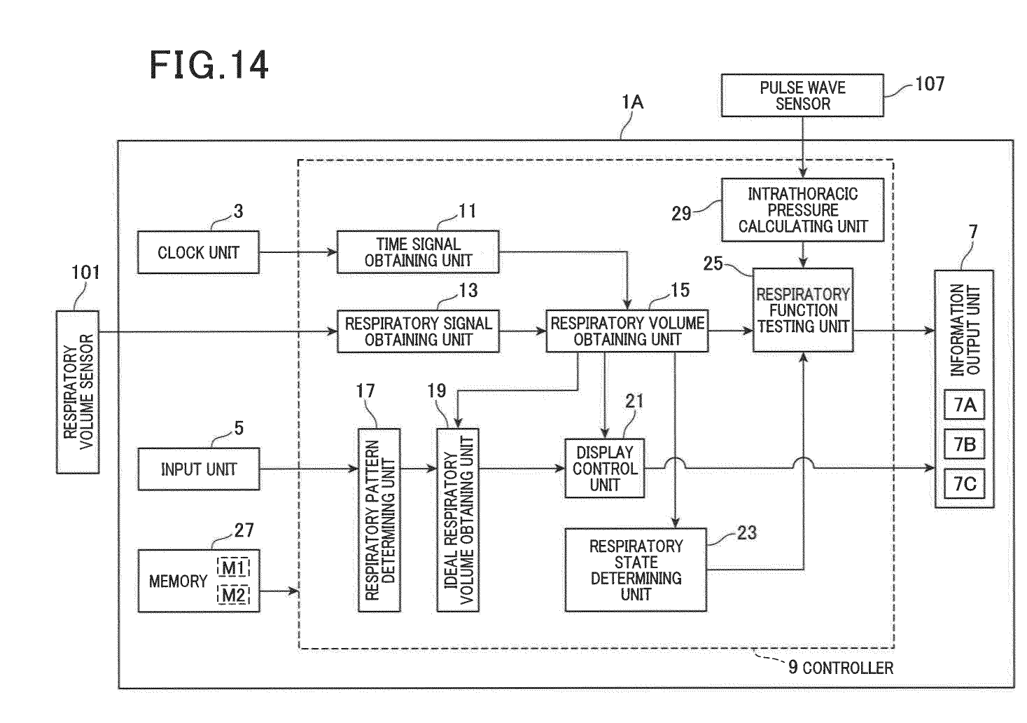

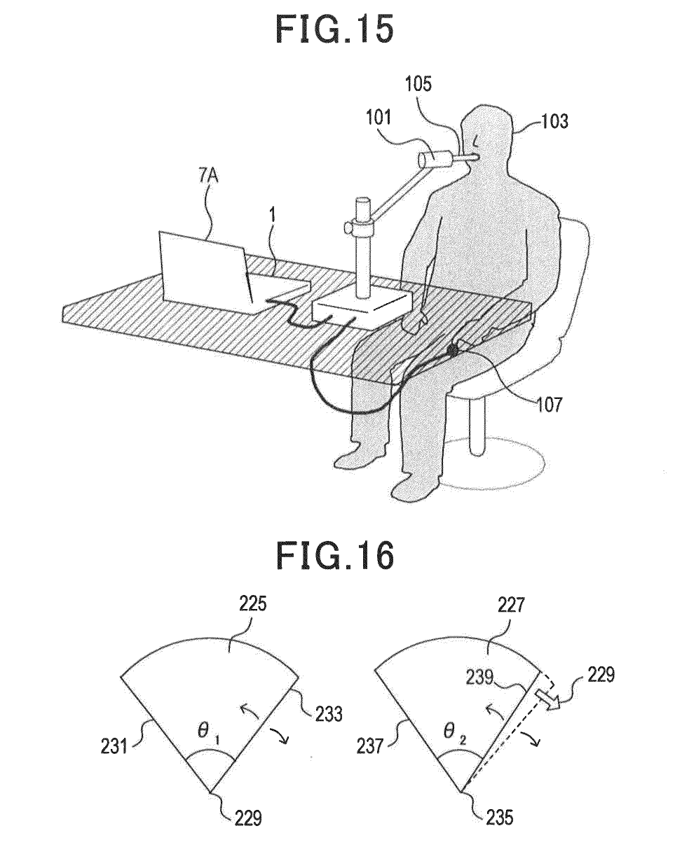

Referring to FIG. 14, the controller 9 further functionally includes an intrathoracic pressure calculating unit 29 operatively coupled to the respiratory function testing unit 25 and communicably coupled to an external pulse wave sensor 107; the pulse wave sensor 107 is capable of measuring a pulse wave signal indicative of pulse waves of a test subject 103 (see FIG. 15).

Specifically, the intrathoracic pressure calculating unit 29 is capable of estimating a value of the intrathoracic pressure of the test subject in accordance with one of the known estimation methods, an example of which is disclosed in Japanese Patent Application Publication No. 2002-355227.

Specifically, as descried above, the intrathoracic pressure calculating unit 29 has the capacity to

1. receive the pulse wave signal sent from the pulse wave sensor 107;

2. measure, based on the pulse wave signal, how the pulse wave signal varies;

3. estimate a value of the intrathoracic pressure of the test subject based on the measured variation of the pulse wave signal; and

4. input the estimated value of the intrathoracic pressure of the test subject to the respiratory function testing unit 25.

Thus, the respiratory function testing unit 25 is capable of measuring the dynamic lung compliance based on variation of the estimated values of the intrathoracic pressure and the aforementioned equation [1].

The specific operations of the controller 9 executing the respiratory-pattern determining task according to the fourth embodiment are substantially identical to those of the controller 9 according to the first embodiment. Similarly, the specific operations of the controller 9 executing the respiration assist task according to the fourth embodiment are substantially identical to those of the controller 9 according to the first embodiment.

Accordingly, the respiratory function test system 1A according to the fourth embodiment achieves substantially the same advantages as those achieved by the respiratory function test system 1.

In addition, the respiratory function test system 1A according to the fourth embodiment is equipped with the intrathoracic pressure calculating unit 29 having the capacity to estimate a value of the intrathoracic pressure of a test subject. This makes it possible to more easily measure the dynamic lung compliance of the test subject.

The present disclosure is not limited to the aforementioned embodiments, and various modifications of the embodiments can be performed within the scope of the present disclosure.

In step S15, the controller 9 can output sounds via the speaker 7B or vibratory motions via the vibrator 7C to guide the subject's respiratory operations in addition to displaying the actual respiratory volume of a test subject (see step S15a in FIG. 4). For example, if the actual respiratory volume of the test subject begins to deviate from the corresponding the ideal respiratory volume, the controller 9 can output sounds via the speaker 7B or vibratory motions via the vibrator 7C; the sounds or vibratory motions encourage the test subject to correct the actual respiratory operation so as to follow the ideal respiratory pattern. An example of such sounds is a sound message of “please breathe in more deeply”.

In step S15, the controller 9 can serve as respiratory state determining unit 23 and the respiratory function testing unit 25 to

1. calculate the deviation of the actual respiratory volume from the corresponding ideal respiratory volume;

2. determine whether the deviation of the actual respiratory volume from the corresponding ideal respiratory volume is greater than a threshold volume in addition to displaying the actual respiratory volume of a test subject (see step S15b in FIG. 4); and

stop measurement of the measurement item corresponding to the selected test item input in step S1 when it is determined that the deviation of the actual respiratory volume from the corresponding ideal respiratory volume is greater than the threshold volume (see YES in step S15b and step S15c in FIG. 4).

Note that, when it is determined that the deviation is equal or lower than the threshold volume (NO in step S15b), the procedure of the respiration assist task goes to step S16.

Instead of the operation in step S15c, in step S15d, the controller 9 can serve as the respiratory function testing unit 25 to

(i) correct a part of an actual respiratory waveform comprising the actual respiratory volumes when it is determined that the deviation of the actual respiratory volume obtained in a cycle of the respiration assist task from the corresponding ideal respiratory volume is greater than the threshold volume; this cycle constitutes the part of the actual respiratory waveform; and

(ii) measure, based on the corrected actual respiratory waveform, a predetermined measurement item corresponding to the test item input from the input unit 5 in step S1.

In the operation in step S15d, the controller 9 can serve as the respiratory function testing unit 25 to

(1) correct the actual respiratory waveform by deleting the part of the actual respiratory waveform when it is determined that the deviation of the actual respiratory volume obtained in a cycle of the respiration assist task from the corresponding ideal respiratory volume is greater than the threshold volume; this cycle constitutes the part of the actual respiratory waveform; and

(2) measure, based on the actual respiratory waveform, a predetermined measurement item corresponding to the test item input from the input unit 5 in step S1.

As a modification of the third embodiment, the controller 9 can display the actual respiratory volume and the ideal respiratory volume in place of the bar graph.

Specifically, in step S26, the controller 9 can display, on the display 7A, a first sector picture 225 serving as a first graphic object; the first sector picture has a center angle θ1 and an area corresponding to the actual respiratory volume obtained in step S23 (see FIG. 16). Specifically, the first sector picture 225 has a first radius 231 with a fixed position on the display 7A, a second radius 233, and a pivot 229 where the first radius 231 and the second radius 233 meet. The position of the second radius 233 is pivotable about the pivot 229, thus changing the center angle θ1 and the area such that the changed center angle θ1 and the changed area are proportional to the actual respiratory volume obtained in step S23.

In step S27, the controller 9 can display, on the display 7A, a second sector picture 227 serving as a second graphic object; the second sector picture 27 has a center angle θ2 and an area corresponding to the ideal respiratory volume at the obtaining time t obtained in step S24 (see FIG. 16). For example, the controller 9 displays the second sector picture 227 at an immediate right side of the first sector picture 225 on the display 7A.

Specifically, the second sector picture 227 has a first radius 237 with a fixed position on the display 7A, a second radius 239, and a pivot 235 where the first radius 237 and the second radius 239 meet. The position of the second radius 239 is pivotable about the pivot 235, thus changing the center angle θ2 and the area such that the changed center angle θ2 and the changed area are proportional to the ideal respiratory volume obtained in step S24.

Thus, in the modification, the center angle θ1 and the area of the first sector picture 225 display the actual respiratory volume obtained at the obtaining time t, and the center angle θ2 and the area of the second sector picture 227 display the ideal respiratory volume at the same obtaining time t.

In addition, in step S28, the controller 9 can display, on the display 7A, a dashed radius positioned at the future ideal respiratory volume obtained in step S25m and display, on the dashed radius, an arrow guide pointer 229. A direction of the arrow guide pointer 229 points out an increase or decrease of the future ideal respiratory volume at the future time t+Δt relative to the ideal respiratory volume at the obtaining time t.

For example, if the future ideal respiratory volume is greater than the ideal respiratory volume at the obtaining time t, the arrow guide pointer 229 located on the right side of the ideal respiratory volume at the obtaining time t is directed rightward. Otherwise, if the future ideal respiratory volume is smaller than the ideal respiratory volume at the obtaining time t, the arrow guide pointer 229 located on the left side of the ideal respiratory volume at the obtaining time t is directed leftward.

The cyclic execution of the respiration assist task according to the modification can result in the center angle θ1 and the area of the first sector picture 225 changing over time depending on variations of the actual respiratory volumes obtained at the respective obtaining times t. The cyclic execution of the respiration assist task according to the modification also can result in the center angle θ2 and the area of the second sector picture 227 changing over time depending on variations of the ideal respiratory volumes at the same obtaining times t. The cyclic execution of the respiration assist task according to the modification further can result in the position of the arrow guide pointer 229 moving rightward or leftward over time depending on variations of the future ideal respiratory volumes at the future times t+Δt. Additionally, the cyclic execution of the respiration assist task according to the modification results in the direction of the arrow pointer 229 showing an increase of decrease of the current ideal respiratory volumes at each obtaining time t.

This enables the test subject to adjust a next actual respiratory time and a next actual respiratory volume after each obtaining time t such that the next actual respiratory volume at the next actual respiratory time after each obtaining time t matches with the ideal respiratory volume at the same time. This results in an improvement of the accuracy of the respiratory function tests to be carried out by the respiratory function test system according to the modification of the third embodiment.

The respiratory function test system 1 according to the first embodiment can be configured not to display the ideal respiratory-pattern waveform 200 on the display 7A. In this modification, displaying the guide pointer 205 makes it possible to guide the respirations of the test subject.

The respiratory function test system 1 according to the first embodiment can be configured to selectively display the ideal respiratory volume at the obtaining time t.

The respiratory function test system 1 according to the first embodiment can be configured to display the ideal respiratory-pattern waveform 200 while deleting the previous portion of the waveform 200 previous to each obtaining time t.

The respiratory function test system 1 according to the first embodiment can be configured not to display the trajectory 203.

The respiratory function test system according to the third embodiment can be configured to display the first and second bars 219 and 221 such that the first and second bars 219 and 221 are aligned vertically.

At least some of the structures of the respiratory function test systems according to the first to fourth embodiments can be combined with each other within the scope of the present disclosure.

While the illustrative embodiments of the present disclosure have been described herein, the present disclosure is not limited to the embodiments described herein, but includes any and all embodiments having modifications, omissions, combinations (e.g., of aspects across various embodiments), adaptations and/or alternations as would be appreciated by those in the art based on the present disclosure. The limitations in the claims are to be interpreted broadly based on the language employed in the claims and not limited to examples described in the present specification or during the prosecution of the application, which examples are to be construed as non-exclusive.

What is claimed is:

1. An apparatus for assisting respirations of a subject in accordance with a predetermined reference respiration pattern defined as a sequence of reference respirations each having a reference respiratory volume for the subject, the apparatus comprising:
a respiration obtaining unit that obtains, at a sampling time, an actual respiratory volume of the subject based on an actual respiration of the subject in accordance with the predetermined reference respiration pattern;
a storing unit that stores variation of the reference respiratory volumes for the subject over time; and
an assisting unit that generates, based on the actual respiratory volume and the variation of the reference respiratory volumes, visual assist information indicative of a respiratory state of the subject relative to the predetermined reference respiration pattern, and provides the visual assist information to the subject.

2. The apparatus according to claim 1, wherein the assisting unit comprises a displaying unit that displays, as the visual assist information, visual information indicative of a relationship between the actual respiratory volume of the subject and at least one of:
a current reference respiratory volume determined based on the variation of the reference respiratory volumes, the current reference respiratory volume corresponding in time to the actual respiratory volume of the subject; and
a future reference respiratory volume determined based on the variation of the reference respiratory volumes, the future reference respiratory volume corresponding to future time, the future time being defined after a lapse of predetermined time from the sampling time.

3. The apparatus according to claim 2, wherein the displaying unit displays a graph indicative of the relationship between the actual respiratory volume and at least one of the current reference respiratory volume and the future reference respiratory volume, the graph having a first axis indicative of time and a second axis indicative of respiratory volume.

4. The apparatus according to claim 2, wherein the graph includes a waveform of the variation of the reference respiratory volumes over time.

5. The apparatus according to claim 2, wherein the displaying unit displays a first graphic object having a variable physical parameter that represents the actual respiratory volume of the subject, and a second graphic object having a second variable physical parameter that represents at least one of the current reference respiratory volume and the future reference respiratory volume.

6. The apparatus according to claim 2, wherein the displaying unit displays a guide pointer indicating the future reference respiratory volume.

7. The apparatus according to claim 2, wherein:
the respiration obtaining unit successively obtains the actual respiratory volume of the subject over time; and
the displaying unit displays, based on the actual respiratory volumes successively obtained by the respiration obtaining unit, a trajectory of variation of the actual respiratory volumes over time.

8. The apparatus according to claim 7, wherein the displaying unit displays a pointer located at a currently obtained actual respiratory volume on the trajectory, the pointer having a direction indicating a direction of the trajectory.

9. The apparatus according to claim 1, wherein the assisting unit generates, based on the actual respiratory volume and the reference respiratory volumes, at least one of sound information and vibration information indicative of the respiratory state of the subject relative to the predetermined reference respiration pattern, and provides the at least one of sound information and vibration information to the subject while providing the visual assist information to the subject.

10. A respiratory function test system for testing a respiratory function of a subject, the respiratory function test system comprising:
a respiration obtaining unit that successively obtains actual respiratory volumes of the subject based on actual respirations of the subject in accordance with a predetermined reference respiration pattern, the predetermined reference respiration pattern being defined as a sequence of reference respirations each having a reference respiratory volume for the subject;
a storing unit that stores variation of the reference respiratory volumes for the subject;
an assisting unit that generates, based on the actual respiratory volumes successively obtained by the respiration obtaining unit and the variation of the reference respiratory volumes, visual assist information indicative of a respiratory state of the subject relative to the predetermined reference respiration pattern, and provides the visual assist information to the subject; and
a respiratory function testing unit that measures, based on the actual respiratory volumes successively obtained by the respiration obtaining unit, at least one measurement item indicative of the respiratory function of the subject.

11. The system according to claim 10, wherein:
the successively obtained actual respiratory volumes include a current respiratory volume that is currently obtained at a current time; and
the assisting unit comprises a displaying unit that displays, as the visual assist information, visual information indicative of a relationship between the current respiratory volume of the subject and at least one of:
a current reference respiratory volume determined based on the variation of the reference respiratory volumes, the current reference respiratory volume corresponding in time to the current respiratory volume of the subject; and
a future reference respiratory volume determined based on the variation of the reference respiratory volumes, the future reference respiratory volume corresponding to future time, the future time being defined after a lapse of predetermined time from the current time.

12. The system according to claim 11, wherein the displaying unit displays a graph indicative of the relationship between the current respiratory volume and at least one of the current reference respiratory volume and the future reference respiratory volume, the graph having a first axis indicative of time and a second axis indicative of respiratory volume.

13. The system according to claim 11, wherein the graph includes a waveform of variation of the reference respiratory volumes over time.

14. The system according to claim 11, wherein the displaying unit displays a first graphic object having a variable physical parameter that represents at least the current respiratory volume of the subject, and a second graphic object having a variable physical parameter that represents at least one of the current reference respiratory volume and the future reference respiratory volume.

15. The system according to claim 10, further comprising:
an input unit that enters a test item representing the at least one measurement item, and a subject information indicative of personal characteristics of the subject; and
a respiratory pattern determining unit that determines one of a plurality of prepared reference respiration patterns based on the test item and the subject information entered by the input unit, the determined one of the plurality of prepared reference respiration patterns being defined as the predetermined reference respiration pattern.

16. The system according to claim 11, further comprising:
a deviation calculating unit that calculates a deviation of the current respiratory volume from the current reference respiratory volume,
wherein the respiratory function testing unit is capable of:
determining whether the calculated deviation is greater than a predetermined threshold volume; and
stopping measurement of the at least one measurement item when it is determined that the calculated deviation is greater than the predetermined threshold volume.

17. A computer program product for an apparatus that assists respirations of a subject in accordance with a predetermined reference respiration pattern defined as a sequence of reference respirations each having a reference respiratory volume for the subject, the computer program product comprising:
a non-transitory computer-readable storage medium; and
a set of computer program instructions embedded in the computer-readable storage medium, the instructions causing a computer to:
obtain an actual respiratory volume of the subject based on an actual respiration of the subject in accordance with the predetermined reference respiration pattern;
store variation of the reference respiratory volumes for the subject; and
generate, based on the actual respiratory volume and the variation of the reference respiratory volumes, visual assist information indicative of a respiratory state of the subject relative to the predetermined reference respiration pattern; and
provide the visual assist information to the subject.
